(19) United States
(12) Patent Application Publication
(10) Pub. No.: US 2026/0207138 A1
(43) Pub. Date: Jul. 23, 2026

(54) SYSTEMS, METHODS AND APPARATUS FOR INFANT MONITORING AND TEMPERATURE-BASED SLEEPWEAR GUIDE

(71) Applicant: Alina SACK, Heatherton (AU)

(72) Inventor: Alina SACK, Heatherton (AU)

(21) Appl. No.: 19/455,138

(22) Filed: Jan. 21, 2026

(30) Foreign Application Priority Data
Jan. 23, 2025 (AU) 2025900179

(51) Int. Cl. A61B 5/00

(52) U.S. Cl. CPC A61B 5/6804; A61B 5/0002; A61B 5/0077; A61B 5/742; A61M 2205/3368

(57) ABSTRACT

A system to monitor an infant and provide a sleepwear guide for the infant comprises a first device to monitor the infant, the first device comprising a camera, a first microphone and a temperature sensor. The system comprises a second device to be used remotely from the first device to receive data wirelessly from the first device, the second device comprising a screen to display images captured by the camera of the first device and a loudspeaker to output audio captured by the first microphone of the first device. The second device further comprises a processor to determine sleepwear to be worn by the infant based on an ambient temperature to be measured by the temperature sensor and to display the determined sleepwear on the screen of the second device.

### CROSS-REFERENCE TO RELATED APPLICATIONS

This application claims priority to Australian Provisional Application No. 2025900179, filed Jan. 23, 2025.

### FIELD OF THE INVENTION

The present invention relates to systems, methods and apparatus for infant monitoring and determining a temperature-based sleepwear guide.

### BACKGROUND TO THE INVENTION

Due to their small size and therefore low heat capacity, it is important to prevent a baby or infant from becoming too hot or too cold. This is typically achieved by clothing or covering the baby or infant in garments, blankets or the like appropriate to the prevailing conditions. This is particularly important at night or at other times when the baby or infant is sleeping because during this time the baby or infant may not be constantly monitored.

However, it can often be difficult for parents, guardians or other caregivers to determine the appropriate garments or blankets to achieve this especially when the prevailing conditions, in particular temperature, are changing, as they often do overnight.

A temperature sensor can determine a current ambient temperature, but the parent, guardian or caregiver must still determine the appropriate garment and/or blanket for the baby or infant. Also, as the temperature changes, the temperature sensor would need to be periodically read by the parent, guardian or caregiver and the appropriate clothing etc. determined.

The weather forecast provides predictions of the outside temperature, but this is not an accurate prediction of the temperature inside.

Furthermore, constant monitoring of a nursery, bedroom or other location in which a baby or infant is sleeping or resting is impractical because the parent, guardian or caregiver is often in another room.

Baby monitors are well known which enable parents, guardians and caregivers to remotely monitor a baby or infant from another location, for example with a camera and/or microphone in a first device which relays images and/or audio of the baby/infant to a second device in the location of the parent/guardian etc. However, baby monitors do not typically monitor temperature and the parent/guardian etc. must still determine the appropriate clothing, blankets etc. for the baby/infant for the current and future temperatures, e.g. overnight.

The reference to any prior art in this specification is not, and should not be taken as, an acknowledgement or any form of suggestion that the prior art forms part of the common general knowledge.

### OBJECT OF THE INVENTION

A preferred object of the present invention is to address or at least ameliorate one or more of the aforementioned problems and/or to provide a useful commercial alternative.

### SUMMARY OF THE INVENTION

Generally, embodiments of the present invention are directed to systems, methods and apparatus to monitor an infant, baby or the like and provide a sleepwear guide for the infant based on a measured temperature to inform parents, guardians or caregivers of the most appropriate sleepwear for the infant, baby or the like.

According to one aspect, a system to monitor an infant and provide a sleepwear guide for the infant is provided, the system comprising:
a first device to monitor the infant, the first device comprising a camera, a first microphone and a temperature sensor;
a second device to be used remotely from the first device to receive data wirelessly from the first device, the second device comprising a screen to display images captured by the camera of the first device and a loudspeaker to output audio captured by the first microphone of the first device;
wherein the second device further comprises a processor to determine sleepwear to be worn by the infant based on an ambient temperature to be measured by the temperature sensor and to display the determined sleepwear on the screen of the second device.

Suitably, the temperature sensor periodically measures the ambient temperature of a room in which the first device is located;
the processor of the second device determines the sleepwear to be worn based on the periodically measured temperature; and
the screen of the second device displays an updated sleepwear determination.

Suitably, the updated sleepwear determination is calculated based on a preset temperature change.

Suitably, the preset temperature change is selected by a user.

Suitably, the sleepwear guide comprises a recommendation of one or more of the following: a Thermal Overall Grade (TOG) rated baby/infant sleeping bag/pouch; a TOG rated baby/infant sleepsuit; one or more layers.

Suitably, the sleepwear guide recommendation is in the form of one or more images and/or text.

Suitably, the sleepwear guide recommendation is colour-coded.

According to another aspect, a method of monitoring an infant and providing a sleepwear guide for the infant is provided, the method comprising:
monitoring the infant by a first device, the first device comprising a camera, a first microphone and a temperature sensor;
receiving wirelessly, by a second device to be used remotely from the first device, data from the first device, the second device comprising a screen to display images captured by the camera of the first device and a loudspeaker to output audio captured by the first microphone of the first device;
determining, by a processor of the second device, sleepwear to be worn by the infant based on an ambient temperature to be measured by the temperature sensor; and
displaying, on the screen of the second device, the determined sleepwear.

The method may further comprise:
periodically measuring, by the temperature sensor of the first device, the ambient temperature of a room in which the first device is located;
determining, by the processor of the second device, the sleepwear to be worn based on the periodically measured temperature; and
displaying, by the screen of the second device, an updated sleepwear determination.

The method may further comprise calculating the updated sleepwear determination based on a preset temperature change.

The method may further comprise receiving a selection of the preset temperature change from a user by the second device.

The method may further comprise displaying the sleepwear guide comprising a recommendation of one or more of the following: a Thermal Overall Grade (TOG) rated baby/infant sleeping bag/pouch; a TOG rated baby/infant sleepsuit; one or more layers.

The method may further comprise displaying the sleepwear guide recommendation in the form of one or more images and/or text.

The method may further display the sleepwear guide recommendation in a colour-coded format.

Suitably, the second device generates an alert when the temperature measured by the temperature sensor in the first device is outside a predetermined temperature range.

Suitably, the predetermined temperature range is set by a user via the second device.

Suitably, the second device generates an alert when the temperature measured by the temperature sensor in the first device is outside a predetermined temperature range, optionally wherein the predetermined temperature range is set by a user via the second device.

Further aspects and/or features of the invention will become apparent from the following detailed description and drawings.

### BRIEF DESCRIPTION OF THE DRAWINGS

Embodiments of the present invention will now be described by way of example only with reference to the accompanying drawings in which like reference numerals refer to like features. In the drawings:

FIG. 1 shows a system to monitor an infant and provide a sleepwear guide for the infant according to one embodiment of the present invention;

FIG. 2 shows a front view of a first device of the system shown in FIG. 1;

FIG. 3 shows a rear view of the first device shown in FIGS. 1 and 2;

FIG. 4 shows a top view of the first device shown in FIGS. 1-3;

FIG. 5 shows a front view of a second device of the system shown in FIG. 1;

FIG. 6 shows a rear view of the second device shown in FIGS. 1 and 4;

FIG. 7 is a screenshot of a user interface displayed on a screen of the second device to access the sleepwear guide and a temperature alert;

FIG. 7A is a screenshot of the user interface displayed on a screen of the second device showing an example of the sleepwear guide;

FIG. 7B is a screenshot of the user interface displayed on a screen of the second device showing a temperature range for the temperature alert;

FIG. 8 shows the screen of the second device displaying sleepwear guide options for a specific measured temperature; and

FIG. 9 is a table showing the sleepwear guide recommendations for a range of temperatures.

Skilled addressees will appreciate that elements in the drawings are illustrated for simplicity and clarity and have not necessarily been drawn to scale. For example, the relative dimensions of some of the elements in the drawings may be distorted and/or some elements may be omitted to help improve understanding of embodiments of the present invention, or some of the drawings may be schematic to best illustrate the invention.

### DETAILED DESCRIPTION OF THE INVENTION

The present invention is directed to systems, methods and apparatus to monitor an infant, baby or the like when sleeping or at rest and to provide a sleepwear guide for the infant based on a measured temperature of the room in which the infant is located to inform users, such as parents, guardians or caregivers of the most appropriate sleepwear recommendations for the infant based on the measured temperature.

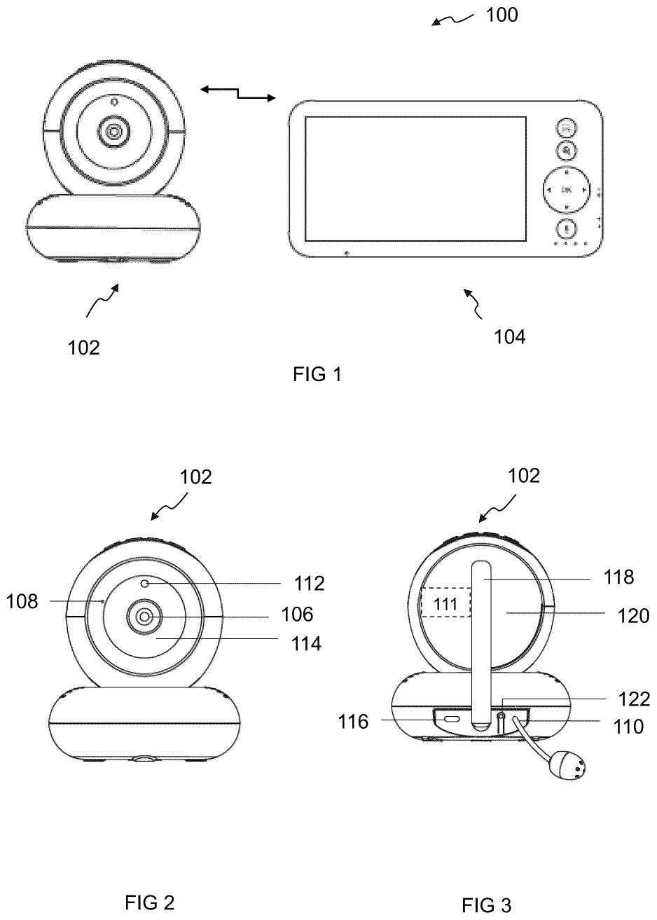

With reference to FIG. 1, according to some embodiments of the present invention, a system 100 to monitor an infant and provide a sleepwear guide for the infant is provided. The system 100 comprises a first device 102 to monitor the infant. The system 100 also comprises a second device 104 to be used remotely from the first device 102 to receive data wirelessly from the first device 102. The first device 102 is paired with the second device 104, and the devices 102, 104 communicate using any secure communications protocol known in the art. For example, frequency-hopping spread spectrum (FHSS) communication can be employed.

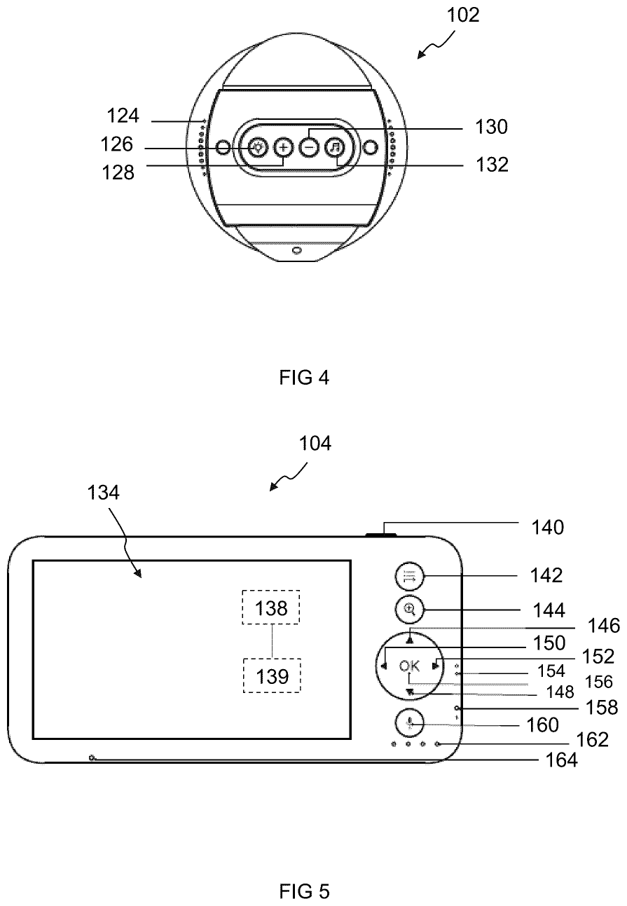

With reference to FIGS. 2, 3 and 4, the first device 102 comprises a camera 106, a first microphone 108 and a temperature sensor 110.

In some embodiments, the first device 102 can also comprise one or more of the following in communication with a processor 111 of the first device 102: a night vision sensor 112; one or more illumination elements 114 such as LEDs; a power input socket 116; an antenna 118; a night light 120; a pairing button 122; a first loudspeaker 124; a first button 126 to operate the illumination elements 114; a second button 128 to increase the brightness of the illumination elements 114 and to increase the volume of the loudspeaker 124; a third button 130 to decrease the brightness of the illumination elements 114 and to decrease the volume of the first loudspeaker 124; a fourth button 132 to start, stop and skip tracks for the playing of a lullaby through the first loudspeaker 124.

In some embodiments, the one or more illumination elements 114 are in the form of eight high intensity infra-red LEDs to enable the camera to detect clear images in low light environments.

In some embodiments, the night light 120 can generate light in a single colour or in multiple colours. For example, the night light can be pressed once for on, pressed further times to cycle between different colours before cycling to an off state. In one embodiment, the night light 120 can be pressed to cycle between the constant colours of yellow, orange or red, or pressed a further time to automatically cycle between these colours.

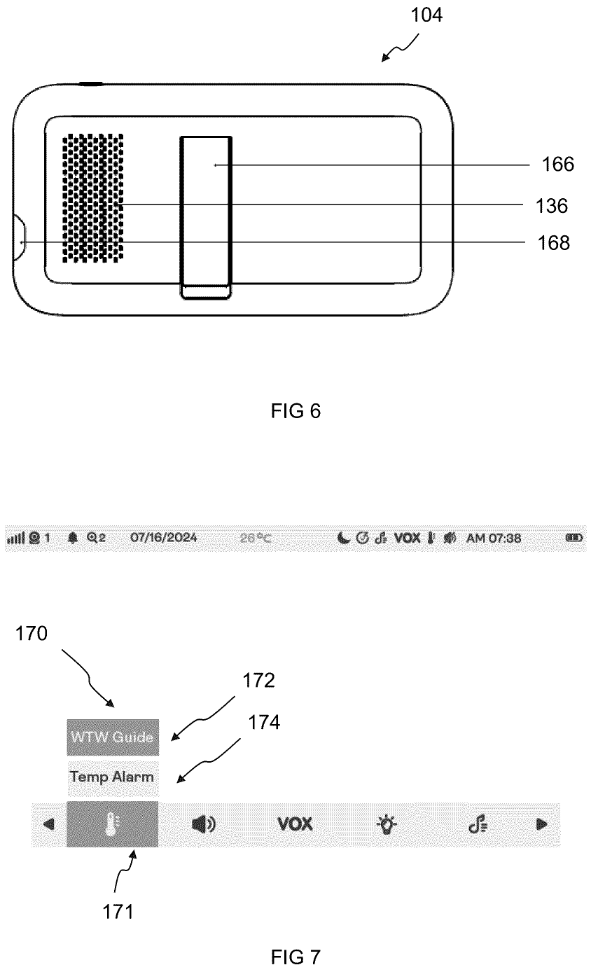

With reference to FIGS. 5 and 6, the second device 104 comprises a screen 134 to display images captured by the camera 106 of the first device 102 and a second loudspeaker 136 to output audio captured by the first microphone 108 of the first device 102.

In some embodiments, the second device 104 can also comprise one or more of the following in communication with a processor 138 of the second device 104: a memory 139; a power on/off button 140; a menu/back button 142; a zoom in/out button 144; a rocker button having: an up action 146 to tilt the camera 106 upwards or move a menu selection up, a down action 148 to tilt the camera 106 downwards or move a menu selection down, a left action 150 to pan the camera 106 left or move a menu selection to the left and a right action 152 to pan the camera 106 to the right or move a menu selection to the right; a power indicator 154 with power level/charging status indicators; a input selection button 156; a monitoring status indicator 158; a talk button 160 to enable users to communicate with the baby/infant; sound level indicators 162; a second microphone 164; a support stand 166; a charging port 168.

The processor 138 of the second device 102 determines sleepwear p recommended to be worn by the infant based on an ambient temperature measured by the temperature sensor 110 and displays the determined sleepwear on the screen 134 of the second device 104.

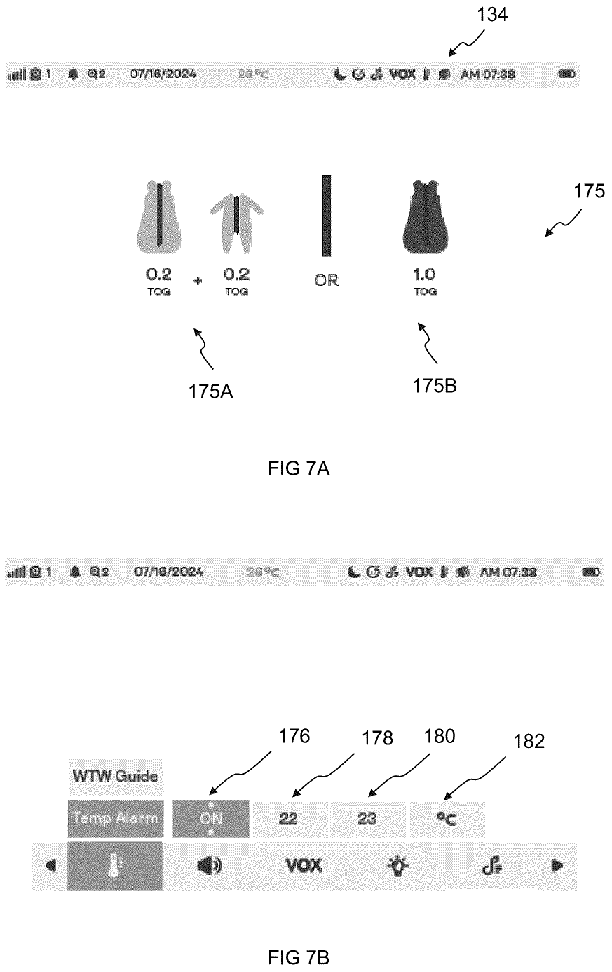

FIG. 7 shows a sample screenshot of the screen 134 of the second device 104 displaying menu options 170 available under a thermometer icon 171. In this example, the menu options 170 include a “what to wear” (WTW) guide icon 172, the selection of which by a user enables the user to navigate to the sleepwear guide.

FIG. 7 also shows a temperature alarm icon 174, the selection of which by a user enables the user to navigate the user interface to a temperature alarm or temperature alert setting menu.

FIG. 7A shows a sample screenshot of the screen 134 of the second device 104 displaying an example of the sleepwear guide 175 determined by the second device 104 based on a temperature of 26° C. (degrees Celsius) of a room in which the baby/infant is located measured by the temperature sensor 110 of the first device 102. In this example, the sleepwear guide 175 comprises two sleepwear recommendations. On the left side of the display, a first sleepwear recommendation 175A comprises images and text showing a combination of a 0.2 Thermal Overall Grade (TOG) baby sleeping bag and a 0.2 TOG long-sleeved sleepsuit. On the right side of the display, a second, alternative sleepwear recommendation 175B comprises an image and text showing a 1.0 TOG baby sleeping bag. The user can select either combination to clothe the baby/infant for the measured temperature of 26° C.

FIG. 7B shows a sample screenshot of the screen 134 of the second device 104 displaying an example of the temperature alarm or temperature alert setting menu. The temperature alarm/alert can be switched on or off by selection of the icon 176. An upper and lower temperature of a temperature range can be set by a user via icons 178, 180, respectively. The units of measurement of the temperature, i.e. Celsius or Fahrenheit, can be set by a user via icon 182. Once the temperature alert/alarm has been activated and the temperature range has been set by the user, or a default temperature range is in place, the second device 104 generates an alert when the temperature measured by the temperature sensor 110 in the first device 102 is outside the preset or predetermined temperature range.

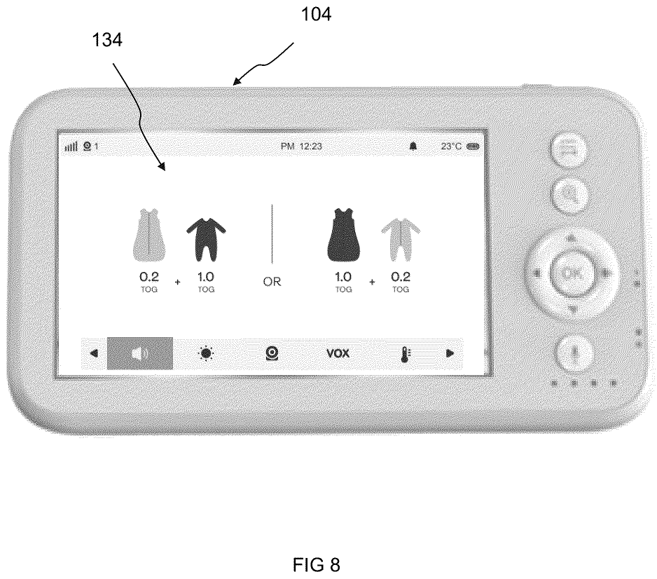

FIG. 8 shows an image of the second device 104 with the screen 134 displaying an example of a sleepwear guide 175 comprising two recommendations. In this example, the measured temperature of the room in which the baby/infant is located is 23° C. (degrees Celsius), and the sleepwear guide comprises two sleepwear recommendations 175A, 175B. On the left side of the display, a first sleepwear recommendation 175A comprises a combination of a 0.2 TOG baby sleeping bag and a 1.0 TOG long-sleeved sleepsuit. On the right side of the display, a second, alternative sleepwear recommendation 175B comprises a combination of a 1.0 TOG baby sleeping bag and a 0.2 TOG long-sleeved sleepsuit. The user can select either combination to clothe the baby/infant for the measured temperature of 23° C.

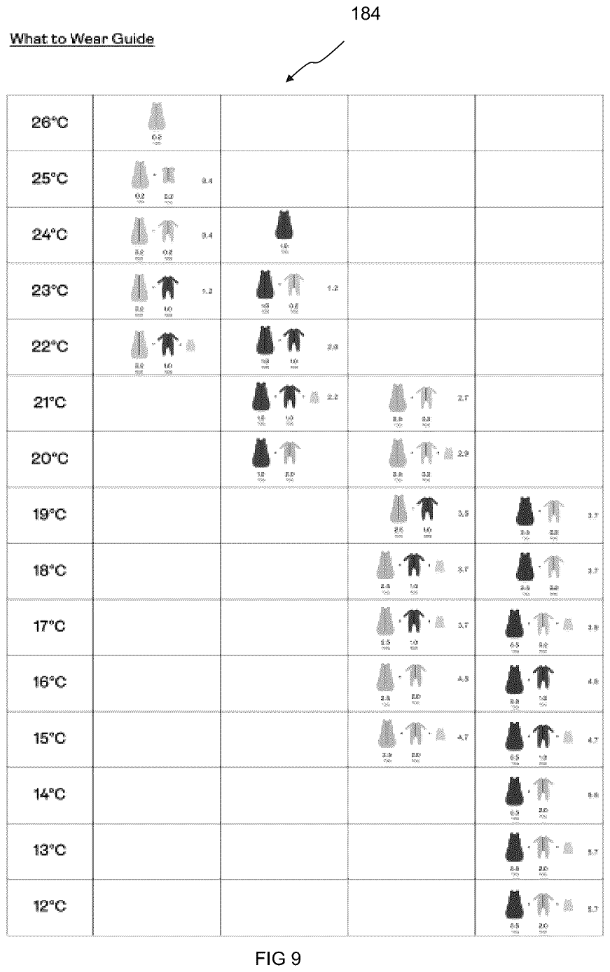

FIG. 9 shows a sleepwear guide 184 in the form of a table comprising the sleepwear guide options or recommendations for a range of temperatures according to some embodiments of the present invention. In this example, the temperature range is for measured ambient temperatures from 12° C. to 26° C., but the present invention is not limited to this particular temperature range. For each temperature, at least one sleepwear recommendation is provided. For at least some temperatures, at least two alternative sleepwear recommendations are provided. It is envisaged that in some embodiments more than two alternative sleepwear recommendations can be provided for at least some temperatures. For each sleepwear recommendation, at least one garment is recommended. For at least some sleepwear recommendations, at least two garments are recommended. For at least some sleepwear recommendations, at least three garments are recommended. For each sleepwear recommendation, a total TOG value may be displayed, which may be achieved with a single garment/layer or a plurality of garments/layers. For example, for the temperature of 23° C., sleepwear guide 184 displays two alternative combinations of garments to achieve a total TOG value of 1.2. For example, for the temperature of 21° C., sleepwear guide 184 displays two alternative combinations of garments. The first option comprises a combination of three garments to achieve a total TOG value of 2.2. The second option comprises a combination of two garments to achieve a total TOG value of 2.7.

In some embodiments, the sleepwear guide comprising at least one sleepwear recommendation comprising at least one recommended garment for each temperature is stored in a lookup table in the memory 139. In some embodiments, the memory 139 stores computer code which, when executed by the processor 138, causes the methods and functionality as described herein to be performed.

In some embodiments, the temperature sensor 110 periodically measures the ambient temperature of a room in which the first device 102 is located. The processor 138 determines the sleepwear to be worn based on the periodically measured temperature and the screen 134 of the second device 104 displays an updated sleepwear determination.

In some embodiments, the updated sleepwear determination is calculated based on a preset temperature change. In some embodiments, the preset temperature change is selected by a user. For example, the user can set a temperature change of 2° C. If a temperature change of 2° C. is measured by the temperature sensor 110, an updated sleepwear determination is calculated by the processor 138 and displayed on the screen 134 of the second device 104.

In some embodiments, the sleepwear guide recommendation is in the form of one or more images and/or text. In the embodiments shown herein, each sleepwear guide recommendation comprises an image of the recommended clothing, such as a baby/infant sleeping bag, a short sleeve sleepsuit, a long sleeve sleepsuit and/or other garment and text in the form of the TOG value for each garment/layer.

In some embodiments, it is envisaged that the sleepwear guide recommendations can be output as audio via the second loudspeaker 136 of the second device 104.

In some embodiments, the sleepwear guide recommendation is colour-coded. For example, a first garment in each recommendation can be in a first colour and a second garment in each recommendation can be in a second colour. The alternative sleepwear guide recommendation for the same temperature can comprise the opposite colour combination to the first sleepwear guide recommendation. Alternatively, contrasting shades of the same colour can be used for the different garments in each sleepwear guide recommendation. Alternatively, first and second garments in a first sleepwear guide recommendation can be in different shades of a first colour and first and second garments in an alternative sleepwear guide recommendation for the same temperature can be in different shades of a second colour.

According to another aspect, embodiments of the present invention are directed to a method of monitoring an infant and providing a sleepwear guide for the infant. The method comprises monitoring the infant by the first device 102, the first device comprising the camera 106, the first microphone 108 and the temperature sensor 110.

The method comprises receiving wirelessly, by the second device 104 to be used remotely from the first device 102, data from the first device 102, the second device 104 comprising the screen 134 to display images captured by the camera 106 of the first device 102 and the second loudspeaker 136 to output audio captured by the first microphone 108 of the first device 102.

The method comprises determining, by the processor 138 of the second device 104, sleepwear to be worn by the infant based on an ambient temperature measured by the temperature sensor 110.

The method comprises displaying, on the screen 134 of the second device 104, the determined sleepwear guide.

In some embodiments, the method further comprises periodically measuring, by the temperature sensor 110, the ambient temperature of a room in which the first device 102 is located.

The method further comprises determining, by the processor 138, the sleepwear to be worn based on the periodically measured temperature.

The method further comprises displaying, by the screen 134, an updated sleepwear determination.

In some embodiments, the method further comprises calculating the updated sleepwear determination based on a preset temperature change.

In some embodiments, the method further comprises receiving a selection of the preset temperature change from a user by the second device.

In some embodiments, the method further comprises displaying the sleepwear guide comprising a recommendation of one or more of the following: a Thermal Overall Grade (TOG) rated baby/infant sleeping bag/pouch; a TOG rated baby/infant sleepsuit; one or more layers.

In some embodiments, the method further comprises displaying the sleepwear guide recommendation in the form of one or more images and/or text.

In some embodiments, the method further comprises displaying the sleepwear guide recommendation in a colour-coded format.

It will be appreciated that the present invention is not limited to the form or visual appearance of the first device 102 and/or the second device 110 shown in the accompanying drawings. For example, the second device 110 could be in the form of a mobile phone, a tablet or other device with a display or other means to communicate the sleepwear guide recommendations.

Hence, embodiments of the present invention address or at least ameliorate the aforementioned problems by providing systems, methods and apparatus to monitor an infant/baby and provide sleepwear guide recommendations for the infant based on the temperature of the room in which the baby/infant is located measured by the temperature sensor in the first device 102. The measured temperature and the sleepwear guide recommendations are provided wirelessly to the second device 104. Thus, parents, guardians or other caregivers are provided with an accurate temperature in the location of the baby/infant and one or more recommendations of sleepwear for the baby/infant based on the measured temperature to prevent the baby/infant from becoming too hot or too cold. The guesswork and stress of trying to determine the appropriate sleepwear for the baby/infant are therefore ameliorated.

The systems, methods and apparatus according to embodiments of the present invention avoid the need for parents, guardians or other caregivers to consult and rely on a weather forecast because the temperature in the room is periodically monitored by the first device 102 and transmitted to the second device 104 where the parents, guardians or other caregivers are located, thus avoiding the need for the parents, guardians or other caregivers to repeatedly return to the location of the baby/infant to measure/read the temperature.

According to some embodiments of the systems, methods and apparatus of the present invention, the processor 138 determines the sleepwear to be worn by the infant/baby based on the periodically measured temperature and displays an updated sleepwear determination on the screen of the second device 104, such that the parents, guardians or other caregivers can change the sleepwear of the baby/infant, if necessary.

Many of the sleepwear recommendations comprise more than one recommendation for a particular measured temperature to provide parents, guardians or other caregivers with a choice of garments/layers in which to clothe the baby/infant.

According to some embodiments, the systems, methods and apparatus of the present invention generate an alert when the temperature measured in the location of the baby/infant is outside a temperature range, which is optionally set by the user.

In this specification, the terms, “first”, “second” etc. are intended to differentiate between different features of the present invention and are not intended to limit the present invention to a particular order of implementation unless the context indicates otherwise.

In this specification, the terms “comprises”, “comprising” or similar terms are intended to mean a non-exclusive inclusion, such that an apparatus that comprises a list of elements does not include those elements solely but may well include other elements not listed.

It will be appreciated that the present invention is not limited to the specific embodiments described herein. Skilled addressees will identify variations from the specific embodiments described herein that will nonetheless fall within the scope of the present invention, which is determined by the following claims.

1. A system to monitor an infant and provide a sleepwear guide for the infant, the system comprising:
a first device to monitor the infant, the first device comprising a camera, a first microphone and a temperature sensor;
a second device to be used remotely from the first device to receive data wirelessly from the first device, the second device comprising a screen to display images captured by the camera of the first device and a loudspeaker to output audio captured by the first microphone of the first device;
wherein the second device further comprises a processor to determine sleepwear to be worn by the infant based on an ambient temperature to be measured by the temperature sensor and to display the determined sleepwear on the screen of the second device.

2. The system of claim 1, wherein:
the temperature sensor periodically measures the ambient temperature of a room in which the first device is located;
the processor determines the sleepwear to be worn based on the periodically measured temperature; and
the screen displays an updated sleepwear determination.

3. The system of claim 2, wherein the updated sleepwear determination is calculated based on a preset temperature change.

4. The system of claim 3, wherein the preset temperature change is selected by a user.

5. The system of claim 1, wherein the sleepwear guide comprises a recommendation of one or more of the following: a Thermal Overall Grade (TOG) rated baby/infant sleeping bag/pouch; a TOG rated baby/infant sleepsuit; one or more layers.

6. The system of claim 5, wherein the recommendation is in the form of one or more images and/or text.

7. The system of claim 5, wherein the recommendation is colour-coded.

8. The system of claim 1, wherein the second device generates an alert when the temperature measured by the temperature sensor in the first device is outside a predetermined temperature range.

9. The system of claim 8, wherein the predetermined temperature range is set by a user via the second device.

10. A method of monitoring an infant and providing a sleepwear guide for the infant, the method comprising:
monitoring the infant by a first device, the first device comprising a camera, a first microphone and a temperature sensor;
receiving wirelessly, by a second device to be used remotely from the first device, data from the first device, the second device comprising a screen to display images captured by the camera of the first device and a loudspeaker to output audio captured by the first microphone of the first device;
determining, by a processor of the second device, a guide to sleepwear to be worn by the infant based on an ambient temperature to be measured by the temperature sensor; and
displaying, on the screen of the second device, the determined sleepwear guide.

11. The method of claim 10, further comprising:
periodically measuring, by the temperature sensor, the ambient temperature of a room in which the first device is located;
determining, by the processor, the sleepwear to be worn based on the periodically measured temperature; and
displaying, by the screen of the second device, an updated sleepwear guide determination.

12. The method of claim 11, further comprising calculating the updated sleepwear determination based on a preset temperature change.

13. The method of claim 12, further comprising receiving a selection of the preset temperature change from a user by the second device.

14. The method of claim 10, further comprising displaying the sleepwear guide comprising a recommendation of one or more of the following: a Thermal Overall Grade (TOG) rated baby/infant sleeping bag/pouch; a TOG rated baby/infant sleepsuit; one or more layers.

15. The method of claim 14, wherein the sleepwear guide recommendation is in the form of one or more images and/or text.

16. The method of claim 14, wherein the sleepwear guide recommendation is colour-coded.

17. The method of claim 10, wherein the second device generates an alert when the temperature measured by the temperature sensor in the first device is outside a predetermined temperature range.

18. The method of claim 17, wherein the predetermined temperature range is set by a user via the second device.
